(19) United States
(12) Patent Application Publication
(10) Pub. No.: US 2024/0079108 A1
(43) Pub. Date: Mar. 7, 2024

(54) Detecting Homologous Recombination Deficiencies (HRD) in Clinical Samples

(71) Applicant: ImmunityBio, Inc., Culver City, CA (US)

(72) Inventors: Andrew Nguyen, Culver City, CA (US); John Zachary Sanborn, Culver City, CA (US); Lucy De Jong, Culver City, CA (US); Stephen Charles Benz, Culver City, CA (US)

(21) Appl. No.: 17/767,615

(22) PCT Filed: Oct. 6, 2020
(86) PCT No.: PCT/IB2020/059348

§ 371 Date: Apr. 8, 2022

Related U.S. Application Data
(60) Provisional application No. 62/913,112, filed on Oct. 9, 2019.

(51) Int. Cl. G16H 20/10; A61K 45/06; G16B 40/00

(52) U.S. Cl. CPC G16H 20/10; A61K 45/06; G16B 40/00

(57) ABSTRACT

Disclosed herein are methods of identifying homologous recombination deficiency (HRD) in omics data, comprising generating a mutational spectrum from omics data; and using the mutational spectrum in a trained model to identify HRD. Further disclosed herein are methods of treating a tumor that has HRD score indicating significant HRD events, comprising: obtaining omics data from a tumor sample and generating a mutational spectrum from omics data; using the mutational spectrum in a trained model to identify HRD in the omics data from the tumor sample; identifying the cancer as likely responsive to treatment with a PARP inhibitor upon determination of HRD; and administering a PARP inhibitor treatment for the tumor upon determination of a high HRD score.

This application claims priority to and the benefit of U.S. Provisional Application No. 62/913,112 filed on Oct. 9, 2019, the entire contents of which is incorporated herein by reference.

### FIELD OF THE INVENTION

The present disclosure relates to systems and methods of omics analysis, and particularly omics analysis of tumor tissue to detect homologous recombination deficiency (HRD).

### BACKGROUND OF THE INVENTION

The background description includes information that may be useful in understanding the present disclosure. It is not an admission that any of the information provided herein is prior art or relevant to the presently claimed invention, or that any publication specifically or implicitly referenced is prior art.

All publications and patent applications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Homologous recombination deficiency (HRD) confers sensitivity to PARP inhibitors (see e.g., Japanese Journal of Clinical Oncology (2019) 49:8, p 703-707), and treatment of ovarian cancers with PARP inhibitors is more likely successful where HRD is found (see e.g., Br J Cancer. 2018 November; 119(11):1401-1409). Similarly, HRD Scores have predicted treatment response to platinum-containing neoadjuvant chemotherapy in patients with triple-negative breast cancer (see e.g., Clin Cancer Res (2016) 22 (15): 3764-75).

Unfortunately, these and other currently used methods to detect HRD are often limited in accuracy and predictive value. As outlined by Matsumoto et al (Japanese Journal of Clinical Oncology (2019) 49:8, p 703-707), the problem of HRD assays is that negative results do not mean lack of response for the efficacy of PARP inhibitors. In some cases, HRD-negative patients also benefit from PARP inhibitors, such as niraparib or rucaparib.

Another problem of the HRD assay is lack of consensus regarding the definition and measurement of each component in the assay: loss of heterozygosity (LOH), telomeric allelic imbalance (TAI), and large-scale state transitions (LST). In further known methods (see e.g., Nature Genetics volume 51, p 912-919 (2019)), machine learning has been employed to detect HRD using signature multivariate analysis. However, such approach is limited to BRCA1/2 mutations and as such still limiting. Indeed, while there are several genetic indicators of HRD, HRD mutational signatures can be independent of single gene mutations. As such, because of the drawbacks listed above, it is difficult to predict which tumor patients would benefit from PARP inhibitors or platinum-based chemotherapy.

As such, even though various systems and methods for HRD detection are known in the art, there is still a need to provide improved systems and methods that allow for detection of HRD from omics data.

### SUMMARY OF THE INVENTION

The inventors have now discovered various systems and methods that allow identification of HRD from omics data, preferably using a trained classifier that recognizes COSMIC mutational spectra associated with HRD.

In one embodiment, provided herein is a method of treating a tumor that has homologous recombination deficiency (HRD) score indicating significant HRD events. The method comprises of obtaining omics data from a tumor sample and generating a mutational spectrum from omics data, and using the mutational spectrum in a trained model to identify HRD in the omics data from the tumor sample. Once HRD is determined in the tumor sample, the tumor/cancer sample is identified as likely responsive to treatment with a PARP inhibitor.

In one embodiment, a PARP inhibitor may be administered as a treatment for the tumor upon determination of a high HRD score. The PARP inhibitor is preferably selected from the group consisting of Olaparib, Rucaparib, Niraparib, Talazoparib, Veliparib, Pamiparib, Rucaparib, CEP 9722, E7016, and 3-Aminobenzamide.

In one embodiment, platinum-based chemotherapy is administered as a treatment for the tumor upon determination of a high HRD score. The platinum-based chemotherapy may be cisplatin, carboplatin or oxaliplatin.

The trained model is preferably generated using machine learning. The machine learning algorithm employs K-means clustering to find and to group optimal clusters in mutational spectra. K-means clustering allows discovery of mutational spectrum show evidence of HRD but do not contain the expected mutations indication HRD.

In one embodiment, the omics data are from a breast cancer sample. In one embodiment, the omics data are from an ovarian cancer sample. Preferably, the omics data do not have germline mutations in BRCA1/BRCA2, CHEK2, PALB2 and/or ATM (signature 3 negative), but have an HRD mutation signature. In one embodiment, the omics data comprises whole genome sequence data.

In one embodiment, the present disclosure provides a method of predicting likely treatment success of a cancer with a PARP inhibitor, comprising: obtaining omics data from a tumor sample and generating a mutational spectrum from omics data; using the mutational spectrum in a trained model to identify HRD in the omics data from the tumor sample; and identifying the cancer as likely responsive to treatment with a PARP inhibitor upon determination of HRD. Most preferably the omics data are whole genome sequencing data. The trained model may be generated using machine learning that employs k-means clustering. The omics data may be from an ovarian cancer or breast cancer sample. The method may further comprise treating the patient with a PARP inhibitor, such as Olaparib, Rucaparib, Niraparib, Talazoparib, Veliparib, Pamiparib, Rucaparib, CEP 9722, E7016, and/or 3-Aminobenzamide. The method may also comprise treating the patient with chemotherapy.

In one embodiment, disclosed is a method of identifying homologous recombination deficiency (HRD) in omics data, comprising: generating a mutational spectrum from omics data; and using the mutational spectrum in a trained model to identify HRD.

Various objects, features, aspects, and advantages will become more apparent from the following detailed description of preferred embodiments, along with the accompanying drawing in which like numerals represent like components.

### BRIEF DESCRIPTION OF THE DRAWING

FIG. 1 depicts an exemplary COSMIC spectrum and determined signatures from the spectrum.

FIGS. 2A and 2B depict PCA reduced data from Signature 3+ BRCA1/2 deficient like samples. FIG. 2A illustrate K-means clustering on BRCA Sig3+ dataset (PCA reduced data). Centroids are marked with white cross. FIG. 2B illustrate the elbow method for optimal k

FIG. 3 depicts exemplary Signature 3 positive clusters.

FIG. 4 depicts exemplary likely pathogenic germline mutations.

FIG. 5 depicts that tumor samples may have a HRD mutation signature without having germline mutations.

FIGS. 6A and 6B depict PCA reduced data from Signature 3 negative data. FIG. 6A illustrate K-means clustering on BRCA Sig3− dataset. Centroids are marked with white cross. FIG. 6B illustrate the elbow method for optimal k.

FIG. 7 depicts exemplary Signature 3 negative clusters.

FIG. 8 depicts exemplary clustering for whole genome sequence breast cancer samples.

FIGS. 9A and 9B depict exemplary mutation spectra for whole genome and exome data.

FIG. 10 depicts an exemplary method of HRD identification/scoring.

FIGS. 11A and 11B depict exemplary variable importance.

### DETAILED DESCRIPTION

The inventors have now discovered that machine learning techniques can be applied to mutational spectra that can then be used to determine mutational signatures. Clustering (e.g., k-means clustering) can then be used to detect optimal clusters to group the data. Notably, such approach has allowed the discovery of different mutational spectra that exhibited evidence of HRD but did not contain the expected mutations that are commonly associated with HRD (e.g., BRCA1/2, CHEK2, PALB2, etc.).

In one embodiment, the instant disclosure provides a method of treating a tumor that has homologous recombination deficiency (HRD) score indicating significant HRD events. The method comprises (a) obtaining omics data from a tumor sample and generating a mutational spectrum from omics data; (b) using the mutational spectrum in a trained model to identify HRD in the omics data from the tumor sample; (c) identifying the cancer as likely responsive to treatment with a PARP inhibitor upon determination of HRD; and (d) administering a PARP inhibitor treatment for the tumor upon determination of a high HRD score.

Genetic abnormalities of the homologous recombination repair (HRR) pathway causes homologous recombination deficiency (HRD) and lead to chromosomal instability. Germline BRCA1/2 mutations, somatic BRCA1/2 mutations, and BRCA gene promotor methylations are well known causes of HRD, but other genetic abnormalities of the HRR pathway could also cause HRD.

While there are several known assays for measuring HRD, such as NCC Oncopanel, FoundationOne, Oncomine, Todai OncoPanel, OncoPrime, MSK-IMPAKT, a negative result in any of these assays does not mean lack of HRD. See Matsumoto et al, Japanese Journal of Clinical Oncology, 2019, 49(8) 703-707. The inventors have solved this problem by using a machine learning omics-based analysis to determine an HRD score.

HRD causes characteristic genomic scar signatures, namely, the loss of heterozygosity (LOH), telomeric allelic imbalance (TAI), and large-scale state transitions (LST). The HRD score is the sum of these scar signature scores. The HRD score correlates with sensitivity to niraparib, which is a PARP inhibitor. As discussed in Akaya et al. a cutoff HRD score ≥42 is indicative for enriched BRCA1/2 mutations for ovarian and breast cancer tumor samples. See Akaya et al. Homologous recombination deficiency status-based classification of high-grade serous ovarian carcinoma. Sci Rep 10, 2757 (2020). As disclosed herein, these patients are likely to be responsive to treatment with a PARP inhibitor.

In one embodiment, omics data obtained from a tumor sample comprises at least one of whole genome sequence information, exome sequence information, transcriptome sequence information, and proteomics information. A COSMIC mutational spectrum is generated from the omics data. The mutational spectrum is then used in a trained model by using machine learning to identify HRD. In one embodiment, machine learning refers to artificial intelligence systems configured to learn from data without being explicitly programmed. Such systems are necessarily rooted in computer technology, and in fact, cannot be implemented or even exist in the absence of computing technology. While machine learning systems utilize various types of statistical analyses, machine learning systems are distinguished from statistical analyses by virtue of the ability to learn without explicit programming and being rooted in computer technology. In one embodiment, the machine learning system is programmed to infer a measurable cell characteristic, out of many different measurable cell characteristics, that has a desirable correlation with the sensitivity data of different cell lines to a treatment. Preferably, the cell characteristic that is measured or inferred by the machine learning system is a mutation in whole genome sequence data of the tumor sample. The machine learning systems used herein are described further in WO2018017467, WO2014210611 etc

In one embodiment, the machine learning algorithm employs K-means clustering to find and to group optimal clusters in mutational spectra. As used herein, the term “cluster” refers to a group of like data points, for example, that are grouped together based on the proximity of the data points to a measure of central tendency of the cluster. For instance, the measure of central tendency may be the arithmetic mean of the cluster, in which case the data points are joined together based on their proximity to the average value in the cluster. K-means clustering refers to a process of grouping like data sets (e.g., gene sequencing data profiles) into groups (e.g., “clusters”) in which each data set belongs to the cluster with the nearest mean. K-means clustering techniques are useful in conjunction with the methods of the invention are known in the art and are described herein.

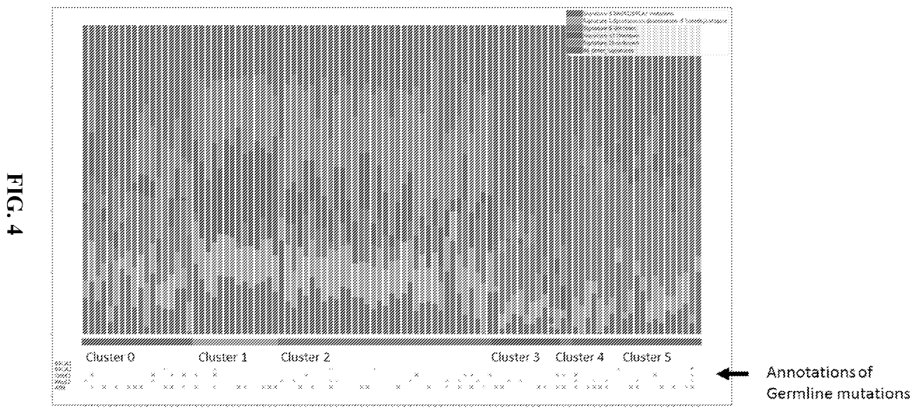

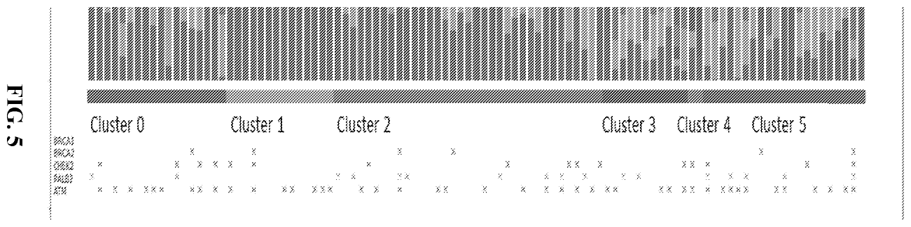

As shown further in FIGS. 4-5, the K-means clustering allowed discovery of mutational spectrum which show evidence of HRD but do not contain the expected mutations indication HRD. In this respect, Catalogue Of Somatic Mutations In Cancer (COSMIC) mutation signatures were used to determine DNA repair defects such as HRD.

The COSMIC mutational signatures are based on an analysis of over 10,952 exomes and 1,048 whole-genomes across 40 distinct types of human cancer. 30 mutational signatures are recognized, and each of these are associated with a cancer type. For example, Signature 1 has been found in all cancer types and in most cancer samples. Signature 2 has been commonly found in cervical and bladder cancers. Signature 3 has been found in breast, ovarian, and pancreatic cancers. Signature 4 has been found in head and neck cancer, liver cancer, lung adenocarcinoma, lung squamous carcinoma, small cell lung carcinoma, and esophageal cancer. Signature 5 has been found in all cancer types and most cancer samples. Signature 6 is most common in colorectal and uterine cancers. Signature 7 has been found predominantly in skin cancers and in cancers of the lip categorized as head and neck or oral squamous cancers. Signature 8 has been found in breast cancer and medulloblastoma. Signature 9 has been found in chronic lymphocytic leukemia and malignant B-cell lymphomas. Signature 10 has been found in colorectal and uterine cancer. Signature 11 has been found in melanoma and glioblastoma. Signature 12 has been found in liver cancer. Signature 13 is common in cervical and bladder cancers. Signature 14 has been observed in four uterine cancers and a single adult low-grade glioma sample. Signature 15 has been found in several stomach cancers and a single small cell lung carcinoma. Signature 16 has been found in liver cancer. Signature 17 has been found in esophagus cancer, breast cancer, liver cancer, lung adenocarcinoma, B-cell lymphoma, stomach cancer and melanoma. Signature 18 has been found commonly in neuroblastoma. Signature 20 has been found in stomach and breast cancers. Signature 21 has been found only in stomach cancer. Signature 22 has been found in urothelial (renal pelvis) carcinoma and liver cancers. Signature 23 has been found in liver cancer. Signature 24 has been observed in a subset of liver cancers. Signature 25 has been observed in Hodgkin lymphomas. Signature 26 has been found in breast cancer, cervical cancer, stomach cancer and uterine carcinoma. Signature 27 has been observed in a subset of kidney clear cell carcinomas. Signature 28 has been observed in a subset of stomach cancers. Signature 29 has been observed only in gingiva-buccal oral squamous cell carcinoma. Signature 30 has been observed in a small subset of breast cancers. In this present disclosure, it should be noted that while the examples (experiments) are on a breast cancer sample having signature 3, the same technique may be used for other cancers as well. Thus, all COSMIC mutational signatures and all of the above different types of cancer tumors are explicitly contemplated herein.

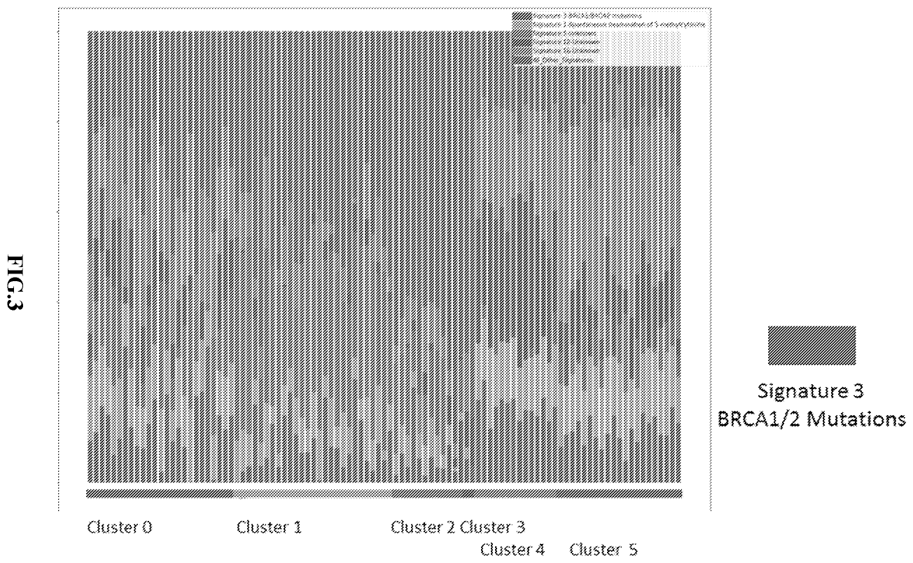

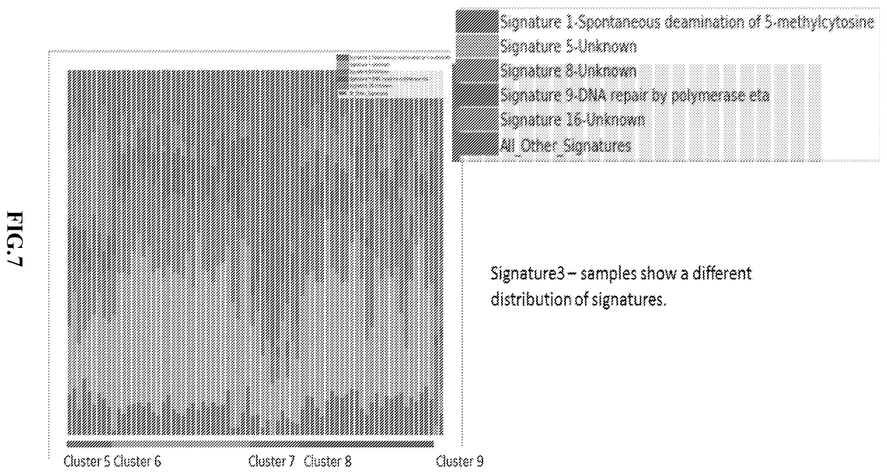

By using the whole genome sequencing approach disclosed herein enabled the discovery of different mutational spectra that exhibited evidence of HRD but did not contain the expected mutations that are commonly associated with HRD (e.g., BRCA1/2, CHEK2, PALB2, etc.). For example, as illustrated in FIGS. 3-5, signature 3 positive samples also contained the mutations expected in signatures 5, 12, and 16. Surprisingly, as illustrated in FIG. 7, signature 3 negative samples (negative for BRCA1/2 mutations) showed a high distribution of mutations expected in signatures 5, 8, 9 and 16, illustrating that sample of these tumor samples have a high HRD score, without having the expected signature 3 mutations.

In breast cancer and ovarian cancer, patients harboring BRCA1/2 mutations exhibit different patterns of clinical behavior and respond to treatment differently. The BRCA gene plays a role in repairing DNA repair via homologous recombination (HR), and mutation of this gene leads to HR deficiency (HRD). HRD can also occur due to other mechanisms, such as germline mutations, somatic mutations and epigenetic modifications of other genes involved in the HR pathway.

As discussed throughout this disclosure, it was surprisingly found that tumor samples that do not have do not have germline mutations in BRCA1/BRCA2, CHEK2, PALB2 and/or ATM (signature 3 negative), may still have high HRD. In these patients, the tumor may be treated with a PARP inhibitor or a platinum-based chemotherapy. Examples of PARP inhibitors contemplated herein comprise Olaparib, Rucaparib, Niraparib, Talazoparib, Veliparib, Pamiparib, Rucaparib, CEP 9722, E7016, and/or 3-Aminobenzamide. Examples of platinum-based chemotherapy contemplated herein comprise cisplatin, carboplatin and oxaliplatin.

Embodiments of the present disclosure are further described in the following examples. The examples are merely illustrative and do not in any way limit the scope of the invention as claimed.

### Example 1

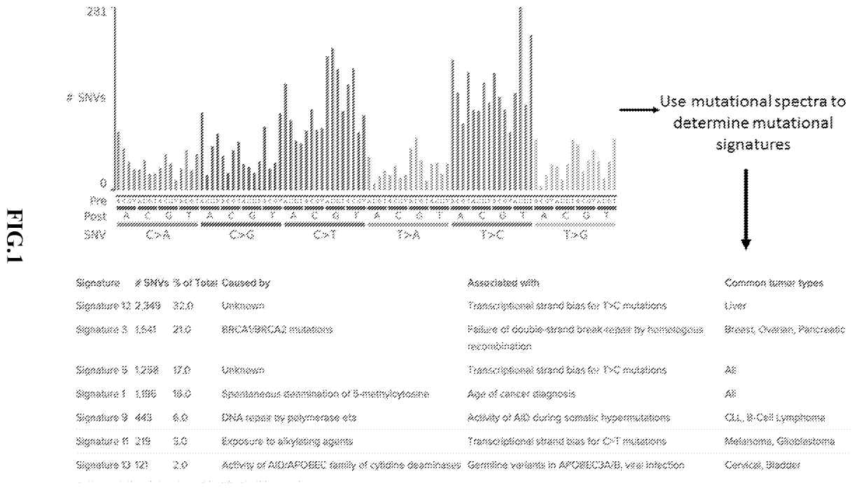

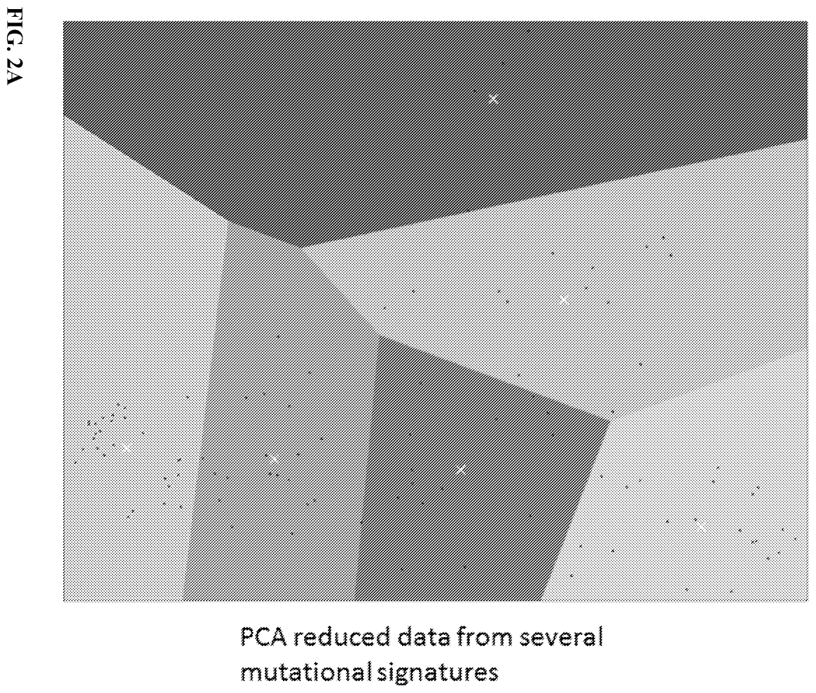

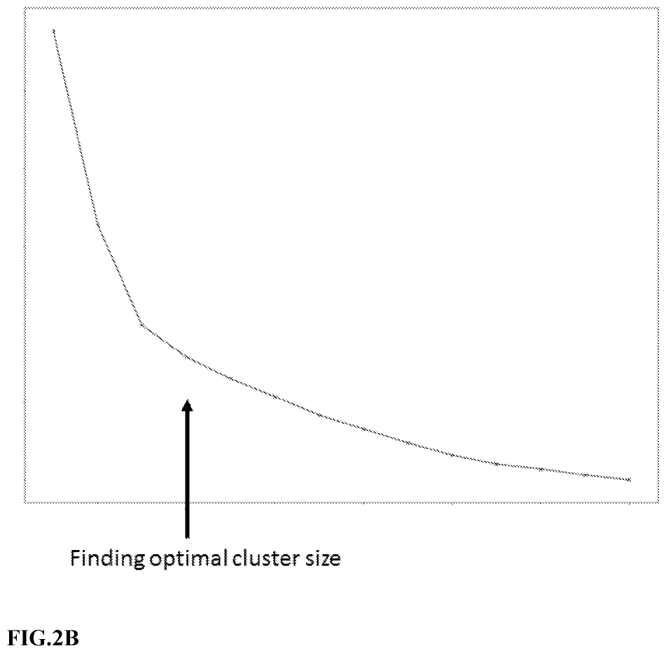

COSMIC mutational signatures/spectra were used to determine mutational signatures and an exemplary spectrum and determined signatures are depicted in FIG. 1. Machine learning with k-means clustering was then employed to find optimal clusters to group the data, which allowed for the discovery of different mutational spectrum that show evidence of HRD but that do not contain the expected mutations indication HRD such as BRCA1/2, CHEK2, PALB2 etc. FIG. 2 depicts an example of such approach using Signature 3+ BRCA1/2 deficient like samples, and FIG. 3 depicts exemplary results for clustering Signature 3 data in which all patient samples showed evidence of defects in the DNA repair machinery. Besides being signature 3 positive, these samples also showed a high distribution of signatures 5, 12, and 16.

FIG. 4 and FIG. 5 illustrate the likely pathogenic germline mutations, and the associated signatures. As illustrated in FIG. 5, 31 of the 101 samples showed no germline mutations in BRCA1/BRCA2, CHEK2, PALB2 or ATM yet they have an HRD mutation signature. Only 6 of the 101 samples had a likely pathogenic BRCA2 germline mutation.

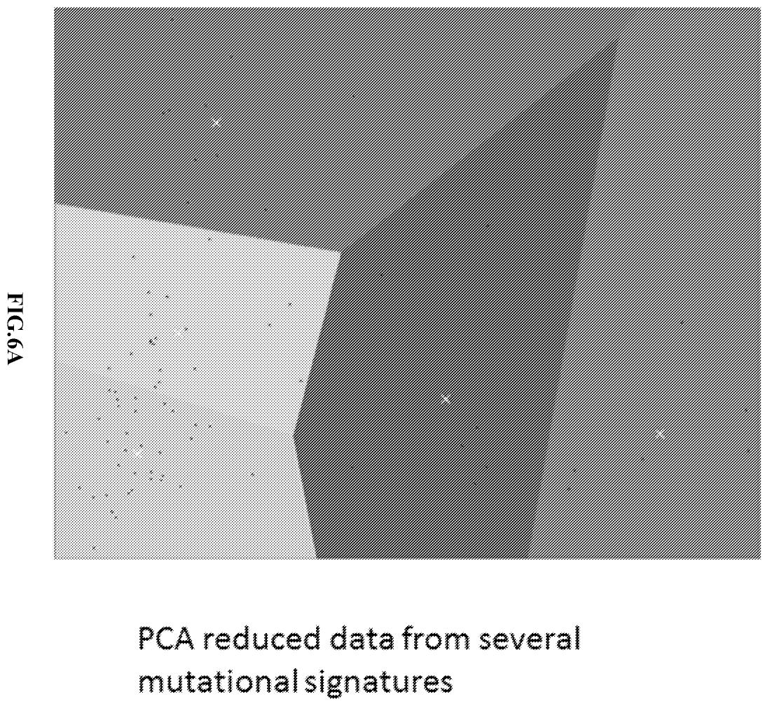

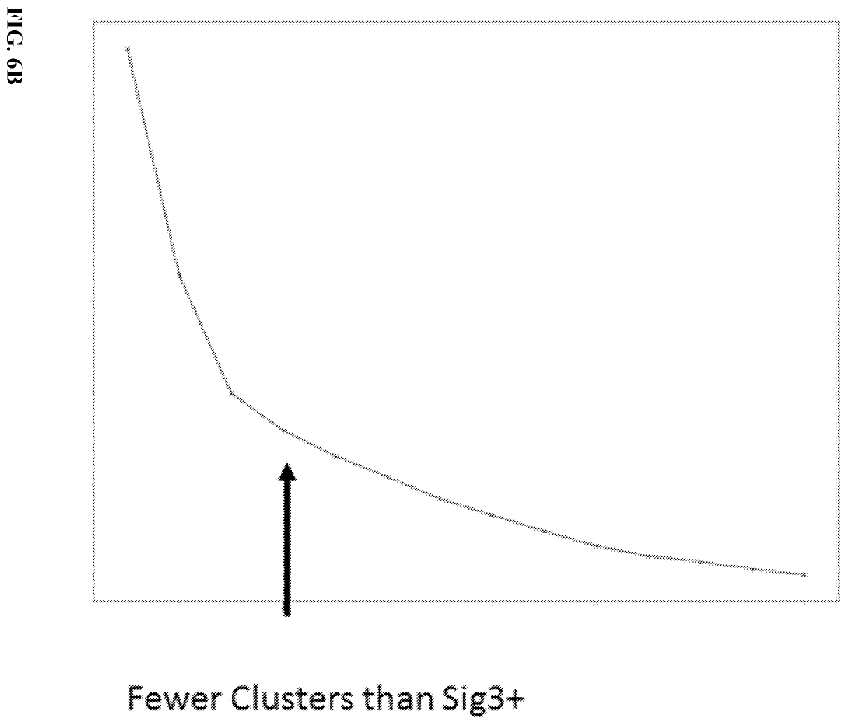

In comparison, samples without Signature 3 presented as shown in FIG. 6, and FIG. 7 exemplarily shows Signature 3 negative clusters. These samples also showed a high distribution of signatures 1, 5, 8, 9, and 16.

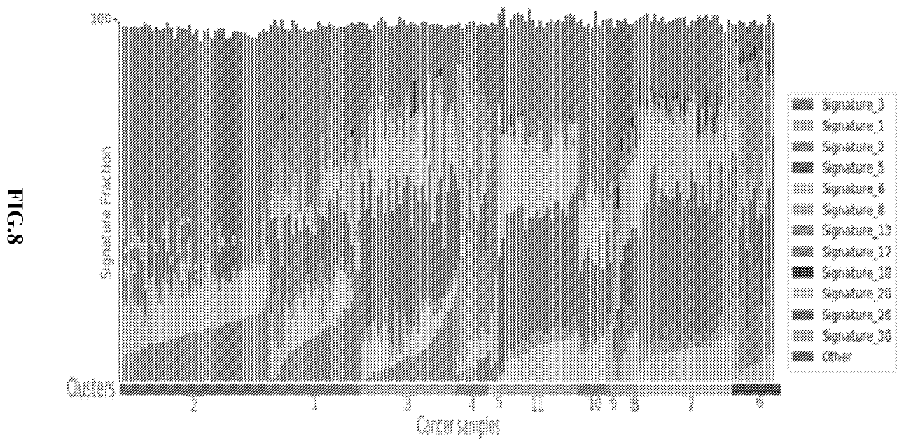

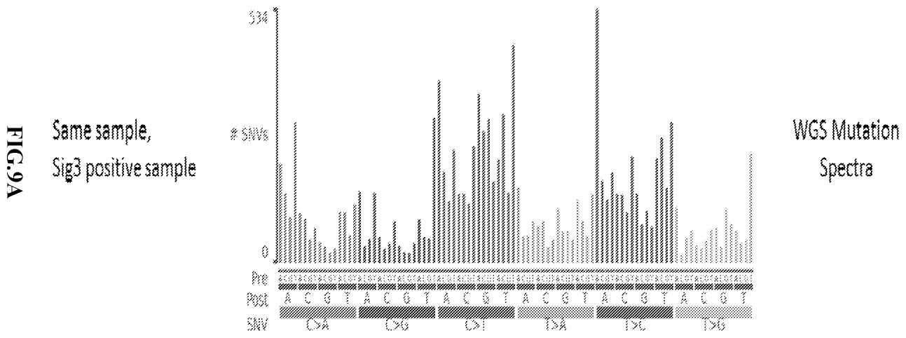

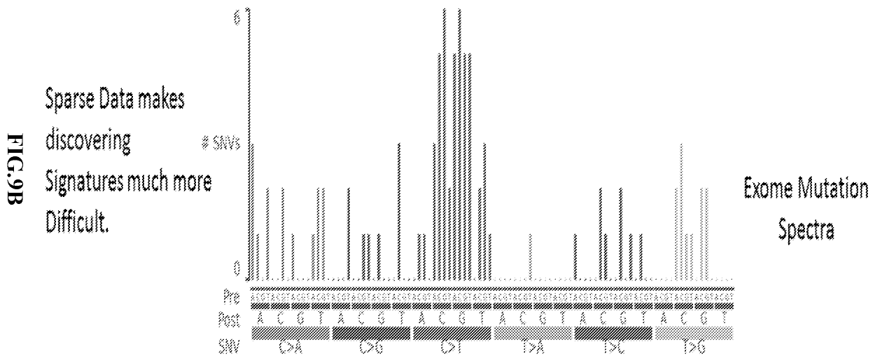

When applied to whole genome sequencing data of breast cancer samples, clustering was observed for Signature 3 positive (n=101) and Signature 3 negative (n=76) samples as can be seen from FIG. 8. Of course, it should be appreciated that mutational spectra can be obtained from data other than whole genome sequencing, and exemplary alternative data include whole exome sequencing (see FIG. 9), albeit the number of data may complicate analysis. Such data can be further refined by analysis of the expression level of the mutated genes as applicable.

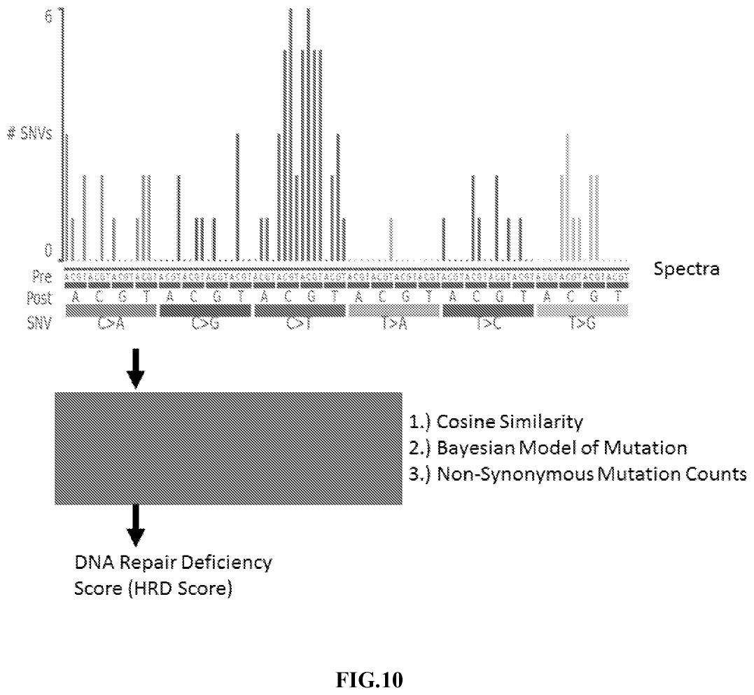

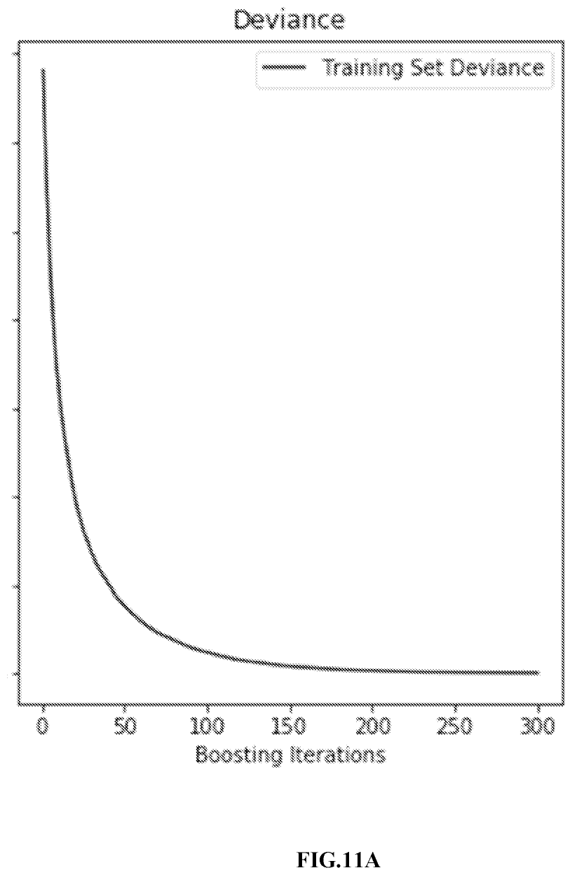

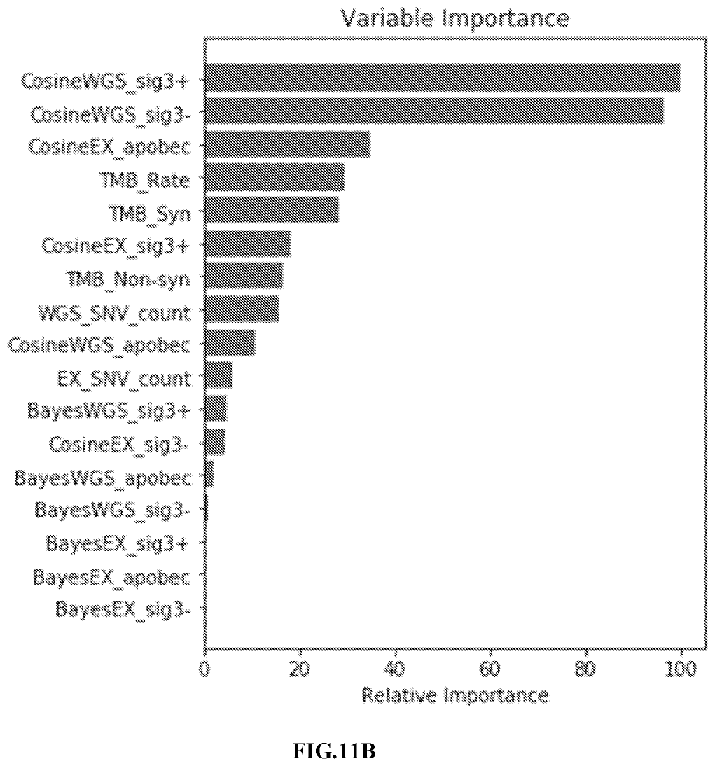

Therefore, it should be noted that machine learning techniques can be employed to train a classifier to recognize mutational spectra. For example, mutational spectra can be reduced to vector space representing mutational counts (e.g., [5,0,0,6,13,25,0,0,2 . . . ]). Alternatively, one could also use similar machine learning techniques that recognize pictures as well as several mathematical functions to compare spectra (e.g., cosine similarity, probability distribution of mutational spectra, etc.). In addition, it should be recognized that multivariate analysis along with ensemble/gradient boosting can be used to derive an HRD Score which also includes non-synonymous mutation count, tumor mutation burden, etc. Therefore, the inventors also contemplate multivariate classifiers as depicted in FIG. 10. Here, the initial model performance provided an average accuracy of ensemble methods predicting HRD of 71%, an average accuracy of cosine metric of 57%, and an average accuracy of probability distribution of 51%. See also FIG. 11. In further contemplated aspects, it should also be recognized that deep nets can be employed to recognize mutational spectra.

Consequently, it should be appreciated that machine learning as presented herein can be employed to generate one or more trained models that will identify HRD from omics data, which can then be used to guide treatment of patients having tumors with HRD. For example, such patients can be treated with PARP inhibitors.

It should be noted that any language directed to a computer should be read to include any suitable combination of computing devices, including servers, interfaces, systems, databases, agents, peers, engines, modules, controllers, or other types of computing devices operating individually or collectively. One should appreciate the computing devices comprise a processor configured to execute software instructions stored on a tangible, non-transitory computer readable storage medium (e.g., hard drive, solid state drive, RAM, flash, ROM, etc.). The software instructions preferably configure the computing device to provide the roles, responsibilities, or other functionality as discussed below with respect to the disclosed apparatus. In especially preferred embodiments, the various servers, systems, databases, or interfaces exchange data using standardized protocols or algorithms, possibly based on HTTP, HTTPS, AES, public-private key exchanges, web service APIs, known financial transaction protocols, or other electronic information exchanging methods. Data exchanges preferably are conducted over a packet-switched network, the Internet, LAN, WAN, VPN, or other type of packet switched network.

As used herein, the term “administering” a pharmaceutical composition or drug refers to both direct and indirect administration of the pharmaceutical composition or drug, wherein direct administration of the pharmaceutical composition or drug is typically performed by a health care professional (e.g., physician, nurse, etc.), and wherein indirect administration includes a step of providing or making available the pharmaceutical composition or drug to the health care professional for direct administration (e.g., via injection, infusion, oral delivery, topical delivery, etc.). It should further be noted that the terms “prognosing” or “predicting” a condition, a susceptibility for development of a disease, or a response to an intended treatment is meant to cover the act of predicting or the prediction (but not treatment or diagnosis of) the condition, susceptibility and/or response, including the rate of progression, improvement, and/or duration of the condition in a subject.

The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the the full scope of the present disclosure, and does not pose a limitation on the scope of the invention otherwise claimed. No language in the specification should be construed as indicating any non-claimed element essential to the practice of the claimed invention.

It should be apparent to those skilled in the art that many more modifications besides those already described are possible without departing from the full scope of the concepts disclosed herein. The disclosed subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

What is claimed is:

1. A method of treating a tumor that has homologous recombination deficiency (HRD) score indicating significant HRD events, comprising:
obtaining omics data from a tumor sample and generating a mutational spectrum from omics data;
using the mutational spectrum in a trained model to identify HRD in the omics data from the tumor sample;
identifying the cancer as likely responsive to treatment with a PARP inhibitor upon determination of HRD; and
administering a PARP inhibitor treatment for the tumor upon determination of a high HRD score.

2. The method of claim 1, wherein the PARP inhibitor is selected from the group consisting of Olaparib, Rucaparib, Niraparib, Talazoparib, Veliparib, Pamiparib, Rucaparib, CEP 9722, E7016, and 3-Aminobenzamide.

3. The method of claim 1, wherein the treatment further comprises platinum-based chemotherapy.

4. The method of any one of the preceding claims, wherein the trained model is generated using machine learning.

5. The method of claim 4, wherein the machine learning algorithm employs K-means clustering to find and to group optimal clusters in mutational spectra.

6. The method of claim 5, wherein the K-means clustering allows discovery of mutational spectrum show evidence of HRD but do not contain the expected mutations indication HRD.

7. The method of claim 1, wherein the omics data are from a breast cancer sample.

8. The method of claim 7, wherein the omics data do not have germline mutations in BRCA1/BRCA2, CHEK2, PALB2 and/or ATM (signature 3 negative) and have a HRD mutation signature.

9. The method of any one of the preceding claims, wherein the omics data comprises whole genome sequence data.

10. A method of predicting likely treatment success of a cancer with a PARP inhibitor, comprising:
obtaining omics data from a tumor sample and generating a mutational spectrum from omics data;
using the mutational spectrum in a trained model to identify HRD in the omics data from the tumor sample; and
identifying the cancer as likely responsive to treatment with a PARP inhibitor upon determination of HRD.

11. The method of claim 10, wherein the omics data are whole genome sequencing data.

12. The method of claim 10, wherein the trained model is generated using machine learning that employs k-means clustering.

13. The method of claim 10 wherein the omics data re from breast cancer.

14. The method of any one of claims 10-13, further comprising treating the patient with a PARP inhibitor.

15. The method of claim 14, wherein the PARP inhibitor comprises Olaparib, Rucaparib, Niraparib, Talazoparib, Veliparib, Pamiparib, Rucaparib, CEP 9722, E7016, and/or 3-Aminobenzamide.

16. The method of any one of claims 11-15, further comprising treating the patient with chemotherapy.

17. A method of identifying homologous recombination deficiency (HRD) in omics data, comprising:
generating a mutational spectrum from omics data;
using the mutational spectrum in a trained model to identify HRD.

18. The method of claim 17, wherein the omics data are whole genome sequencing data.

19. The method of claim 17, wherein the trained model is generated using machine learning that employs k-means clustering.

20. The method of any one of claims 17-19 wherein the omics data are from breast cancer.
